(19) United States
(12) Patent Application Publication
(10) Pub. No.: US 2008/0189829 A1
(43) Pub. Date: Aug. 14, 2008

(54) Device For Venous and/or Lymphatic Insufficiency of Lower Limbs

(75) Inventor: Maria Antonietta Fusco, Avellino (IT)

Correspondence Address: LEFFERT JAY & POLGLAZE, P.A., P.O. BOX 581009, MINNEAPOLIS, MN, 55458-1009, US

(73) Assignee: KS Italia s.a.s. di Ambrosone Mario & C., Avellino (IT)

(21) Appl. No.: 11/662,988

(22) PCT Filed: Sep. 16, 2005
(86) PCT No.: PCT/IB2005/053050

§ 371 Date: Jan. 16, 2008

(30) Foreign Application Priority Data
Sep. 17, 2004 (IT) RM2004 A 000444

(51) Int. Cl. A61F 13/08

(52) U.S. Cl. 2/239

(57) ABSTRACT

Device (1) for the therapy or prevention of venous and/or lymphatic insufficiencies of lower limbs, comprising: a pair of elastocompression tights (2) for venous and/or lymphatic insufficiency of lower limbs; and a plurality of elastic bulges (3-9) for stimulating the venous and lymphatic circulation, which are filled-up with discreet particles made of caoutchouc, is integral to the tights (2) and is arranged at a portion (20) thereof which is in contact with the foot sole when the tights themselves (2) are worn.

The present invention relates to a device for the therapy or prevention of venous and/or lymphatic insufficiencies of lower limbs.

As it is well known for the persons skilled in the art, in the foot sole human beings have a structure called Lejan's venous sole. This is a tangle of blood capillaries indovated in loose fatty tissue, enriched with fundamental substance, chondroitin sulfate and jaluronic acid, which capillaries are squashed during deambulation and, like a sponge full of blood, they are squeezed by a simple mechanical squashing action, imparting to blood a centripetal push rising towards the heart.

In order to correct or prevent possible deficiencies of the venous return mentioned above and also of the lymphatic return, different types of plantars are known which can be inserted inside shoes and which aim at stimulating selected areas of the foot sole for reflexogenic purposes.

Nevertheless, the known plantars tend to slide or, however, to move with respect to the foot sole, especially when they are worn together with woman stockings or short socks. This determines a not correct positioning of the plantar itself with respect to the foot and a consequent vanification of the effect thereof.

Also due to the mentioned drawbacks, the therapeutic result obtainable by means of a plantar can be extremely limited, especially with respect to the variety of possible vascular and lymphatic pathologies that can affect the person's lower limbs.

To treat and prevent such vascular and/or lymphatic pathologies, the so-called elastocompression socks are often used, which have a structure so as to facilitate the venous and/or lymphatic return. However, also the results obtainable with such socks are often limited and incomplete as far as the treatable pathologies are concerned.

Therefore, the technical problem set and solved by the present invention is to provide a device for the prevention or therapy of venous and/or lymphatic insufficiencies of lower limbs allowing obviating to the drawbacks mentioned above with reference to the known art.

Such problem is solved by a device according to claim 1.

Preferred features of the present invention are included in the dependent claims thereof.

In the present context, the term “sock” is to be understood in its broadest sense, including any piece of clothing apt to cover part of or the whole foot and, in case, also part of or the whole lower limb of the person and, in particular, stockings, tights, socks and small socks for men, shank socks and so on.

The present invention provides some important advantages. The main advantage consists in that the synergy between the structure of the elastocompression sock and the bulge(s) incorporated therein allow obtaining a combined therapeutical result which greatly helps the person and allows preventing or treating the vascular and/or lymphatic pathologies thereof in a more effective way. Furthermore, the risk that the bulges can move with respect to their correct position with respect to the foot is avoided.

Other advantages, features and application modes of the present invention will be evident from the following detailed description of some embodiments thereof, shown by way of example and not for limitative purposes. Reference will be made to the figures of the enclosed drawings, wherein:

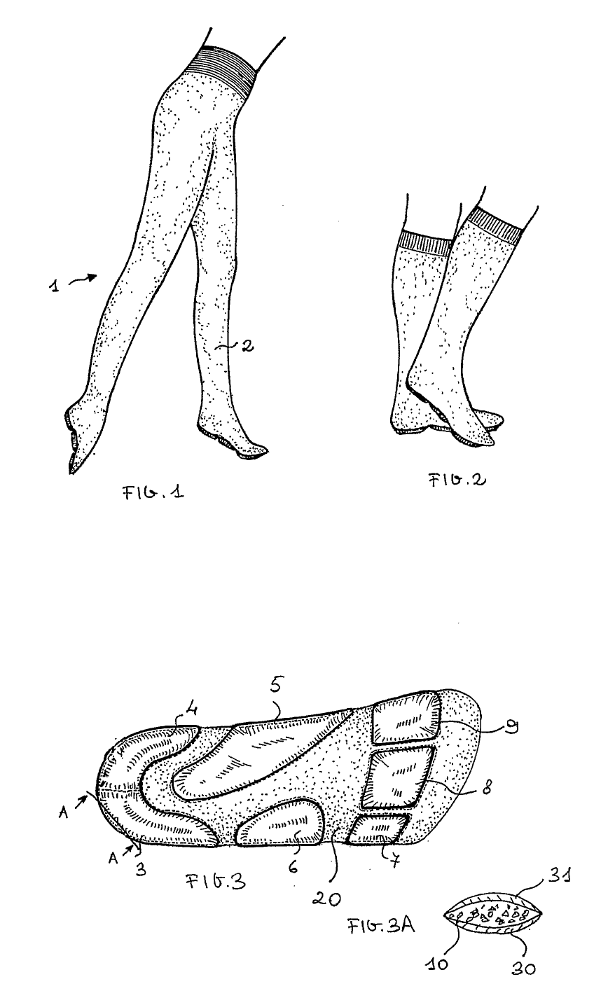

FIG. 1 shows a perspective view of a first embodiment of the device according to the present invention, when worn by a person;

FIG. 2 shows a perspective view of a second embodiment of the device according to the present invention, when worn by a person;

FIG. 2 shows a partial top view of the device of FIG. 1 or 2;

FIG. 3A shows a cross-sectional view of the device, taken according to line A-A of FIG. 3; and

FIG. 4 shows a partial top view of an additional embodiment of the device according to the present invention.

By referring to FIG. 1, a device for the prevention or therapy of a venous and/or lymphatic insufficiency of lower limbs according to an embodiment of the invention is designated as a whole with 1.

The device 1 includes an elastocompression sock 2 having a structure implemented according to known modes to facilitate the venous and lymphatic return of lower limbs. In the present example, the sock 2 is a pair of tights. The tights 2 wholly wrap-up the lower limbs and the feet of the person wearing it.

As it is better visible in FIG. 2, seven elastic bulges, designated with 3, 4, 5, 6, 7, 8 and 9, respectively, are incorporated in the sock 2, arranged at a portion 20 of the sock 2 apt to adhere to the foot sole of the person and fixed with said portion. Said bulges 3-9 are apt to stimulate the venous and/or lymphatic circulation of the lower limbs and, in particular, the venous and/or lymphatic return. Preferably, the bulges 3-9 are arranged at reflexiological areas of the foot sole, as shown indeed in the figures.

As it is known to the person skilled in the art, the plantar reflexotherapy consists in stimulating determined places of the foot sole, substantially corresponding to the muscle insertions of the so-called intrinsic muscles for maintaining the plantar archs, by which it is possible to influence the general physiology of the human body for therapeutic purposes and, in particular, the venous and lymphatic return.

In general, the bulges 3-9 are apt to perform a proprioceptive, esteroceptive, pressoceptive and/or reflexogenic stimulation for correcting the vascular and/or lymphatic disturbs and to this purpose they are arranged at esteroceptors, proprioceptors, pressoceptors and/or any other reflexological place of the foot sole. Such proprioceptos, esteroceptors, pressoceptors and reflexologic places of the surface, articular and deep type are positioned substantially at the muscle insertions of the foot sole.

In particular, as schematically shown in the figures, a first bulge can be arranged at the big toe's adductor, a second bulge at the big toe's short flexor, a third bulge at the short flexor of the fifth toe, a fourth bulge at the adductor of the fifth toe, a fifth bulge at the supinator wedge, a sixth bulge at the pronator wedge and a seventh bulge at the big toe's adductor.

Of course, embodiment variants can provide bulges arranged in positions corresponding to any other place of esteroceptive, pressoceptive and reflexological stimulation of the foot sole, in association or not with the seven proprioceptive places mentioned above.

In particular, an additional preferred embodiment is shown in FIG. 4. As it can be seen in said figure, the bulges are arranged just at the areas of the sole foot belonging to the so-called Lejan's venous sole, and specifically a first bulge 11, preferably with rounded shape, is arranged at the junction of the collecting veins of the toes, a second bulge 12 extends along the medial margin of the foot and it is arranged on the anatomic seat of the medial plantar venous plexus and a third bulge 13 is placed onto the calcaneal area and at the side margin of the foot to convey the venous and lymphatic flow towards the medial venous plexus.

As shown in FIG. 3A, in the implementations described sofar each bulge, for example the one designated with 3, includes a first lower layer 30 and a second upper layer 31, with equal shape and sizes, overlapped and coupled so as to implement an alveolus.

The first thin and about 1-mm-thick layer 30 is made of a non-toxic and substantially non-allergic rubber material, having appropriate long-lasting peculiarities. As rubber material, a material of natural origin and in particular a Para rubber or caoutchouc is preferred. Of course, a person skilled in the art will be able to appreciate that any other type of non-allergic material with equivalent mechanical properties could be utilized. In particular, synthetic rubbers, for example silicone rubbers, could be used.

The second layer 31, thin too, is made of a material soft to the touch and suitable to the contact with the foot sole, in particular of the type which is of chamois or velvet type to the touch. A tissue of natural origin is preferred, and, in particular, alcantara. Embodiment variants can then provide the use of a tissue or a rolled sheet of a material suitable to guarantee the foot transpiration and a sufficient contact comfort.

The first layer 30 and the second layer 31 are fastened therebetween, for example glued, at the bulge periphery.

The alveoli perimeter can be reinforced with respective linear sewings which can be used also to fasten the alveoli themselves to the sock 2.

In the present embodiment, each bulge 3-9 further provides a filling with elastic discreet particles 10 preferably made of a substantially non-allergic rubber material. For a better stimulation effect, preferably such particles 10 have a substantially irregular shape and have facetings and edges.

The particles 10, which can be manufactured by properly cutting in small cubes the Para rubber or caoutchouc sheet used to make up the first layer 30, have, each one, cross sizes of about 1 mm. Alternatively, a compound of 1-mm-thick natural caoutchouc with one silicone grain, with an irregular shape too and with a thickness of about 2 mm, or even of silicone grain only can be provided.

A person skilled in the art will understand that, alternatively or together with said discreet particles 10, alveoli can be filled-up with any other type of elastic material, for example a material like gel, which results to be simple to manipulate and to be inserted in the alveoli.

The thickness of each resulting bulge 3-9 is preferably about 3 mm.

Each alveolus can have an opening that can be closed in order to allow the filling-in thereof by means of a cannula or a similar device.

Then, it will be understood that the bulges 3-9 have an elastic structure as a whole apt to perform the stimulation mentioned above of the foot sole.

In particular, the shape, thickness and elasticity of each bulge can be selected so as to obtain a correct reflexotherapic stress of the proprioreceptors, esteroceptors, pressoceptors and reflexological places in general of the foot sole. In particular, such parameters of the bulges 3-9 are selected based upon the therapeutic directions related to the patient who has to wear the device 1. Furthermore, the device 1 according to the present invention can be manufactured in various sizes. The alveoli can remain empty until, based upon medical prescriptions, they are filled-up with an adequate quantity of elastic particles 10.

Similarly, even the compression intensity of the sock 2 can be adapted to the patient's specific requirements. Regarding this latter aspect, aids as precautionary measure, with an elastocompression sock from about 15 to 25 den, therapeutic aids for mild problems, with an elastocompression sock from about 30 to 50 den, and therapeutic aids for more important venous insufficiency, with an elastocompression sock from about 50 to 70 den, can be provided.

In terms of pressor action exerted onto the person's limb, an aid as precautionary measure can be provided, with an elastocompression sock apt to exert a mild compression of about 20 mm Hg, a therapeutic aid for mild problems, with an elastocompression sock apt to exert a medium compression equal to about 30 mm Hg, and a therapeutic aid for more important venous insufficiency, with an elastocompression sock apt to exert a strong compression equal to about 40-50 mm Hg.

It will be appreciated that so-constructed bulges are able to provide to the patient an optimum reflexotherapic stimulation. In particular, they are apt to create a greater thickness and consequently a stronger pressure in the area of the mesofoot during deambulation. Such stimulation can be kept unaltered for a long period of time even longer than the life year.

The persons skilled in the art, then, will comprise that in presence of venous and lymphatic insufficiency with decrease in the speed of both blood and lymphatic circulation in the legs due to dilatation of the veins and to a lesser squashing of the plantar venous sole, the device of the invention brings about considerable benefit and this thanks to the increase in the mechanical squashing caused by the elastic bulges positioned in the sock together with a discrete elasticity of the sock itself apt to control the dilatation of the venous walls.

Furthermore, the device 1 does not suffer from particular wear and it is always comfortable to wear. Furthermore, it can be washed and it is easy to be used and maintained.

The materials of which it is made guarantee the non-allergenicity and the non-toxicity of the plantar.

Of course, the device is susceptible of several embodiments alternative to the one described sofar. For example, it can have even one single bulge. Furthermore, the bulges can be implemented directly as continuous extension of the sock's tissue, which in case can be made thicker locally to receive the filling material, instead of having the structure with double layer described above. Moreover, the sock can incorporate a real plantar having said bulge(s).

FIG. 2 shows, then, an additional embodiment of the invention device, wherein the sock is a shank sock or a short sock for men.

The present invention has been described sofar by referring to preferred embodiments. It is to be meant that other embodiments belonging to the same inventive core may exist, all comprised within the protective scope of the herebelow reported claims.

1. Device apt to be worn during deambulation for the therapy or prevention of venous and/or lymphatic insufficiencies of lower limbs, comprising:
an elastocompression sock for venous and/or lymphatic insufficiency of lower limbs;
characterized in that it further comprises
a plurality of elastic bulges apt to stimulate the venous and lymphatic circulation, which bulges are fixed with said sock and arranged at a portion thereof which goes into contact with the foot sole when the sock itself is worn at selected reflexological areas of the foot sole itself.

2-19. (canceled)

20. Device according to claim 1, wherein said elastocompression sock is a pair of tights.

21. Device according to claim 1, wherein said elastocompression sock is a short sock.

22. Device according to claim 1, wherein said elastocompression sock is a shank stock.

23. Device according to claim 1, wherein said elastocompression sock is a woman stocking.

24. Device according to claim 1, wherein said reflexological areas of the foot sole are selected in a group comprising areas corresponding to: big toe's adductor, big toe's short flexor, short flexor of the fifth toe, adductor of the fifth toe, supinator wedge, pronator wedge and big toe's adductor.

25. Device according to claim 1, wherein said bulge(s) are arranged at Lejars's venous sole of the foot sole.

26. Device according to claim 1, wherein said bulges are arranged at selected areas in a group comprising: the junction of the toes' collecting veins, the anatomical seat of the medial plantar venous plexus and the calcaneal area at the side edge of the foot.

27. Device according to claim 1, wherein said bulge(s) has(have) a lower layer and an upper layer apt to contact with the foot sole, wherein between said layers a filling material is or can be interposed.

28. Device according to claim 27, wherein said lower layer is made of rubber.

29. Device according to claim 27, wherein said upper layer is made of alcantara.

30. Device according to claim 27, wherein said filling material is formed by elastic discreet particles.

31. Device according to claim 30, wherein said particles have a substantially rounded shape.

32. Device according to claim 29, wherein said particles comprise particles made of caoutchouc.

33. Device according to claim 29, wherein said particles comprise silicone grains.

34. Device according to claim 1, wherein said sock incorporates a plantar having said bulges.

35. Device according to claim 1, wherein said sock has a thickness comprised between about 15 and 25 den.

36. Device according to claim 1, wherein said sock has a thickness comprised between about 30 and 50 den.

37. Device according to claim 1, wherein said sock has a thickness comprised between about 50 and 70 den.
